(19) United States
(12) Patent Application Publication
(10) Pub. No.: US 2025/0325810 A1
(43) Pub. Date: Oct. 23, 2025

(54) THERAPEUTIC DEVICE AND METHOD OF MANUFACTURING THEREOF

(71) Applicant: SHENZHEN NUON MEDICAL EQUIPMENT CO., LTD., ShenZhen (CN)

(72) Inventors: ALAIN DIJKSTRA, Amstelveen (NL); LI XIANG, Shenzhen (CN); Tang Yuanzhou, Shenzhen (CN); Chen Kunjie, Shenzhen (CN); Feng Jianxin, Shenzhen (CN)

(73) Assignee: SHENZHEN NUON MEDICAL EQUIPMENT CO., LTD., Shenzhen (CN)

(21) Appl. No.: 18/788,223

(22) Filed: Jul. 30, 2024

(30) Foreign Application Priority Data
Apr. 22, 2024 (CN) 2024208401517
May 8, 2024 (CN) 2024209909521
Jun. 28, 2024 (CN) 2024215195779

(51) Int. Cl. A61N 1/36; A61N 1/04; A61N 5/06

(52) U.S. Cl. CPC A61N 1/36014; A61N 1/0476; A61N 5/0616; A61N 2005/0644; A61N 2005/0651

(57) ABSTRACT

Embodiments of the present invention provide a therapeutic applicator and a method of providing therapeutic treatment to a user thereof. The therapeutic applicator has an applicator for scooping and applying a cosmetic at one end and at least one stimulation element at the second end. The applicator end of the therapeutic applicator comprises a heating element, a cooling element or a Peltier element. The therapeutic application may further comprises one or more sensor to monitor and measure one or more parameters of skin of the user, such as cosmetic temperature, skin temperature, treatment time, skin Ph. The therapeutic applicator may further comprise elements for pre-treatment of the user's skin before applying the cosmetic.

### TECHNICAL FIELD

The present invention relates generally to recreational and therapeutic devices. More specifically, the present invention relates to therapeutic devices that use combinational therapies to provide alternate therapies for skin-related disorders.

### BACKGROUND ART

Combinational alternative therapies involving light therapy, heating, cooling, vibratory massage, electrotherapy, etc., have been used for quite some time to provide benefits such as pain relief, skin rejuvenation, wound healing, and improvement of overall mental health. However, such combinational therapies require several distinct devices in a professional setup, such as a spa, a gymnasium, or a salon.

In recent years, alternate therapy technology has flourished in the field of beauty and medicine. Phototherapy, a form of alternate therapy, uses specific wavelengths of light to act on the skin, stimulating the vitality and function of cells, improving skin problems, and promoting overall health. Phototherapy beauty technology is usually performed with professional beauty equipment in professional centers. For skin care at home, some home beauty equipment that uses phototherapy are available in the market. The currently available solution generally uses a spoon to apply cosmetics, such as an eye cream or a facial cream, and a phototherapy equipment to perform phototherapy on face that accelerates the absorption of cosmetics on the face, thereby improving the effect of physical therapy.

However, the equipment used for application and therapy effect are usually separate and must be carried out separately during physical therapy. This makes it inconvenient for a user to carry multiple equipment, which affects the therapeutic effects.

### OBJECTS OF THE INVENTION

Some of the objects of the invention are as follows:

An object of the present invention is to provide a therapeutic device with a relatively very small footprint in size and power consumption.

Another object of the present invention is to provide a simple, economical therapeutic device with low production costs.

Another object of the present invention is to provide a therapeutic device that eliminates the need to carry multiple equipment for performing alternate therapy.

Another object of the present invention is to provide a therapeutic device capable of providing a combination of distinct therapies such as light therapy, heating, cooling, vibratory massage, electrotherapy, etc.

Another object of the present invention is to provide a therapeutic device that is convenient to hold during usage of the device and application of several different therapies.

Another object of the present invention is to provide a therapeutic device that can be operated remotely using a user computing device.

### SUMMARY OF THE INVENTION

According to a first aspect of the present invention, a therapeutic applicator is provided. The therapeutic applicator comprising: a scoop body having a first end and a second end, the first end has an applicator for scooping and applying a cosmetic; a housing at the second end of the scoop body; a slotting unit in the housing at the second end of the scoop body, the slotting unit comprises a support base, a PCB, at least one stimulation element in electrical connection with the PCB, and a power source.

In one embodiment of the invention, the at least one stimulation element is selected from a group consisting of Light Emitting Diodes (LEDs), lasers, heating elements, cooling elements, vibration elements, electrodes, micro-current element, and combinations thereof.

In one embodiment of the invention, the scoop body comprises one or more sensors to sense the pH and to monitor one or more parameters of the skin of a user.

In one embodiment of the invention, the therapeutic applicator is in communication with an external communication device and communicates one or more parameters to the external communication device, the external communication device determines a treatment regime for corresponding portion and communicates the treatment regime to the therapeutic applicator.

In one embodiment of the invention, the therapeutic applicator further comprising a microcontroller to activate and control the at least one stimulation element based on the treatment regime determined for the corresponding portion.

In one embodiment of the invention, the first end of the scoop body further comprises a heating element, a cooling element, a Peltier element, or a combination thereof.

In one embodiment of the invention, the first end of the scoop body comprises a brush, a rotating brush, or an abrasive element to treat the skin before applying the cosmetic.

In one embodiment of the invention, the therapeutic applicator further comprising a front cover unit having a first end and a second end, the first end of the front cover unit locks into the first end of the scoop body to cover the slotting unit and the housing.

In one embodiment of the invention, the second end of the front cover unit is curved.

In one embodiment of the invention, the therapeutic applicator is turned on when a user holds the device, and the first end of the scoop body is in contact with the cosmetic or skin of the user.

In one embodiment of the invention, the therapeutic applicator further comprising a charging needle installed on the scoop body.

In one embodiment of the invention, the center of the scoop body is circular in shape for holding by a user, and the diameter of the center is less than the width at the first end and the second end of the scoop body.

According to a second aspect of the present invention, a therapeutic applicator is provided. The therapeutic applicator comprising: a scoop body having a first end and a second end, the first end has an applicator for scooping and applying a cosmetic; a housing at the second end of the scoop body; at least one stimulation element in the housing at the second end of the scoop body; wherein the first end of the scoop body comprises a thermal regulating element.

In one embodiment of the invention, the at least one stimulation element is selected from a group consisting of Light Emitting Diodes (LEDs), lasers, heating elements, cooling elements, vibration elements, electrodes, micro-current element, and combinations thereof.

In one embodiment of the invention, the thermal regulating element comprises a heating element, a cooling element, a Peltier element, or a combination thereof.

In one embodiment of the invention, the scoop body comprises one or more sensors to sense the pH and to monitor one or more parameters of the skin of a user, the therapeutic applicator communicates the one or more parameters of the skin to an external communication device.

In one embodiment of the invention, the external communication device determines a treatment regime for the corresponding portion and communicates the treatment regime to the therapeutic applicator.

In one embodiment of the invention, the therapeutic device further comprising a microcontroller to activate and control the at least one stimulation element based on the treatment regime determined for the corresponding portion.

In one embodiment of the invention, the therapeutic device further comprising a micro-current element.

According to a third aspect of the present invention, a method for providing skin therapy to a user is provided. The method comprising: providing a therapeutic applicator having an applicator for scooping and applying a cosmetic at a first end, and at least one stimulation element at the second end; applying the cosmetic to a user's skin; using the stimulation element to provide therapeutic treatment to the user's skin.

In one embodiment of the invention, the at least one stimulation element is selected from a group consisting of Light Emitting Diodes (LEDs), lasers, heating elements, cooling elements, vibration elements, electrodes, micro-current element, and combinations thereof.

In one embodiment of the invention, the therapeutic treatment includes but is not limited to phototherapy, cooling, heating, laser treatment, massage, and induction of micro-current.

In one embodiment of the invention, the method further comprising: sensing the pH of the skin and monitoring one or more parameters of the skin by using one or more sensors.

In one embodiment of the invention, the skin of the user is pre-treated by a brush, a rotating brush, or an abrasive element in the applicator before the cosmetic is applied to the skin.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of the specification, the phrase “communication network” refers to a group of several connected devices including computing devices (such as desktops, mobile handheld devices, tablet PCs, notebooks, etc.), local and remotely located servers (such as web servers, application servers, database servers, Application Program Interface (API) servers, load balancers, compute nodes, and the like), routers, antennas, modems, multiplexers, demultiplexers, and the like. In that regard, the aforementioned connected devices may be able to exchange data signals through wired and/or wireless means as per several combinations of several different communication protocols such as 802.11 (Wi-Fi), 802.3 (Ethernet), Bluetooth, NFC, ZigBee and 3GPP protocols such as HSPA, HSDPA, LTE, GSM, CDMA, WLL and the like.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, when an element is referred to as being “fixed to” or “disposed to” another element, it may either directly on another element or indirectly on that other element. When a component is said to be “connected” or “connected to” another component, it may be directly connected to another component or indirectly connected to other component on the piece.

In the context of the specification, the terms “first”, “second” and “third” are only used for descriptive purpose and does not implicate the relative importance or to implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates a front perspective view of a therapeutic device in accordance with an embodiment of the present invention;

FIG. 2 illustrates an exploded view of the therapeutic device of FIG. 1;

FIG. 3 illustrates a stimulation component of the therapeutic device in accordance with an embodiment of the present invention;

FIG. 4 illustrates a support base of the stimulation component in accordance with an embodiment of the present invention;

FIG. 5 illustrates a cover of the support base in accordance with an embodiment of the present invention.

FIG. 6 illustrates a back portion of the therapeutic device having a receiving groove in accordance with an embodiment of the present invention.

FIG. 7 illustrates a front cover unit of the therapeutic device in accordance with an embodiment of the present invention.

FIG. 8 illustrates a light emitting plate on the front cover unit of the therapeutic device in accordance with an embodiment of the present invention.

FIG. 9 illustrates a therapeutic applicator with a micro-current element in accordance with an embodiment of the present invention.

FIG. 10 illustrates a charging station for the therapeutic applicator in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a “therapeutic device” or a “therapeutic applicator”. The therapeutic applicator comprises a scoop body having a first end and a second end, the first end has an applicator for scooping and applying a cosmetic; a housing at the second end of the scoop body; a slotting unit in the housing at the second end of the scoop body, the slotting unit comprises: a support base, a Printed Circuit Board (PCB) and at least one stimulation element in electrical connection with the PCB and a power source connected to the PCB.

Several additional functionalities may also be added to the therapeutic device. For example, the applicator may have one or more sensors to sense the pH and monitor one or more parameters of the skin of a user. Moreover, activation and control of specific stimulation elements may be based upon feedback received from the one or more sensors, such as pressure sensors (for ascertaining contact with a body portion of the user) and temperature sensors (for maintaining the temperature of heating and/or cooling elements within permissible limits) about several parameters, such as temperature of cosmetic, skin temperature, treatment time, skin pH. Electrical power to operate the therapeutic device may be provided through an onboard rechargeable battery that may be charged using a power cable. The therapeutic device may also have a heating element, a cooling element, or a Peltier element to treat the cosmetic/liquid. The therapeutic applicator may have additional components for the pre-treatment of the skin, such as a brush, a rotating brush, or an abrasive element to treat the skin before applying the cosmetic. Moreover, the therapeutic device may turned ON when a user holds the device and the applicator part is in contact with the cosmetic or skin of the user. The therapeutic device can also be controlled by a user computing device communicating with the therapeutic device over a communication network through a communication interface built into the therapeutic device.

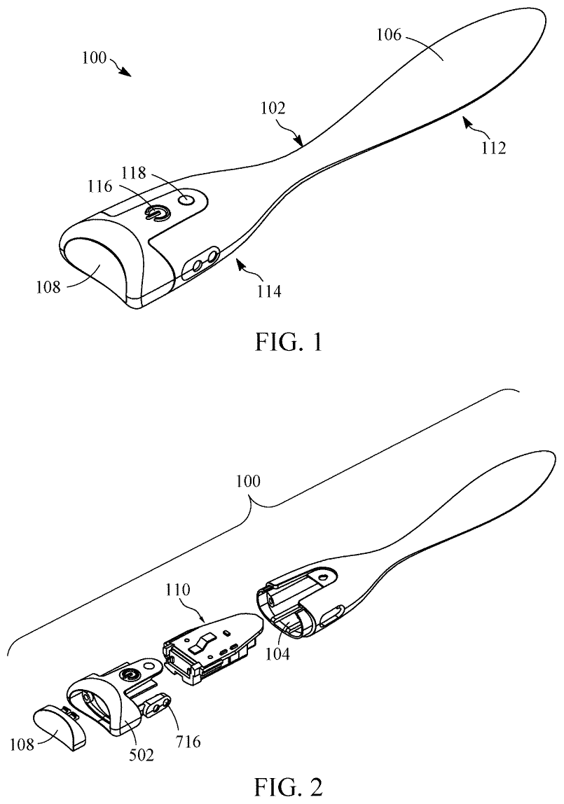

Several embodiments of the present invention will now be described in detail with references to FIGS. 1-8.

FIG. 1 illustrates a front perspective view of a therapeutic device 100 (hereinafter also referred to as “the device 100”) in accordance with an embodiment of the present invention. FIG. 2 illustrates an exploded view of the therapeutic device of FIG. 1. Referring to FIG. 1 and FIG. 2, the therapeutic applicator comprises two sections: a stimulation element section 114, and a scoop section 112. The therapeutic applicator comprises a scoop body 102, one or more stimulation elements, and a light emitting plate 108. The scoop body 102 can be in any geometrical or non-geometrical shape. A first end of the scoop body 102 is provided with an applicator portion 106. The shape of the end with the applicator portion 106 is a flat curvilinear triangle. The applicator portion 106 is used for scooping a cosmetic and applying it over the user's skin, thereby replacing the waste caused by taking the cosmetic directly with hands. The second end of the scoop body 102 has a housing 104 for accommodating one or more stimulation elements and other components of the therapeutic device. The housing 104 shown in FIG. 1 and FIG. 2 is rectangular in shape. However, the housing 104 can be in any corresponding geometrical or non-geometrical shape to accommodate the one or more stimulation elements and other components. A front cover unit 502 having a first end and the second end is provided, the first end of the front cover unit 502 covers the one or more stimulation element and the housing 104 of the scoop body. The housing 104 has one or more slots into which corresponding section on the first end of the front cover unit 502 insert, and the front cover unit 502 gets locked into the scoop body 102. A light emitting plate 108 is installed on the second end of the front cover unit 502 such that the light emitting plate 108 is arranged in the light emitting direction of the one of the stimulation element.

In an embodiment of the present invention, the one or more stimulation elements are selected from a group consisting of Light Emitting Diodes (LEDs), lasers, heating elements, cooling elements, vibration elements, electrodes, micro-current elements, and a combination thereof.

In an embodiment, the therapeutic applicator 100 comprises a scoop section 112 and a stimulator section 114. The width first increases and then decreases in the direction away from the stimulator section 114, and the scoop section 112 is away from the stimulator section 114. The width of the scoop section 112 is smaller than the width of the end of the scoop section close to the stimulator section 114, and the thickness of the scoop section 112 is oriented away from the stimulation section 114. The thickness of the stimulator section 114 first increases and then decreases. The thickness of the end of the scoop section 112 away from the stimulation section 114 is smaller than that of the scoop section 112. The width of the stimulation section 114 gradually increases in the direction away from the scoop section 112. The width of the end of the scoop section 112 is greater than the width of the scoop section 112, and the thickness of the stimulation section away from the scoop section 112. The thickness of the scoop section 112 gradually increases, and the thickness of the end of the stimulation section 114, which is close to the scoop section 112, is greater than that of the scoop section 112. The width of the stimulations section 114 is greater than the maximum thickness of the scoop section 112. The center of the scoop body is configured to have a thinner structure with a smaller width, which is convenient for hand positioning and cleaning. The end farthest away from the stimulation section 114 has the smallest width and thickness, making it easy to scoop the cosmetic at the bottom of the cosmetic bottle.

The one or more stimulation elements are embodied in a slotting unit 110. The slotting unit 110 is installed in the housing 104 present at the second end of the scoop body 102. The housing 104 in the second end of the scoop body 102 accommodates the slotting unit 110 with one or more stimulation elements, a main circuit board or a Printed Circuit Board (PCB) in electrical connection with the one or more stimulation elements and a power source.

In one embodiment of the invention, the scoop body comprises one or more sensors to sense the pH and monitor one or more parameters of the skin of a user.

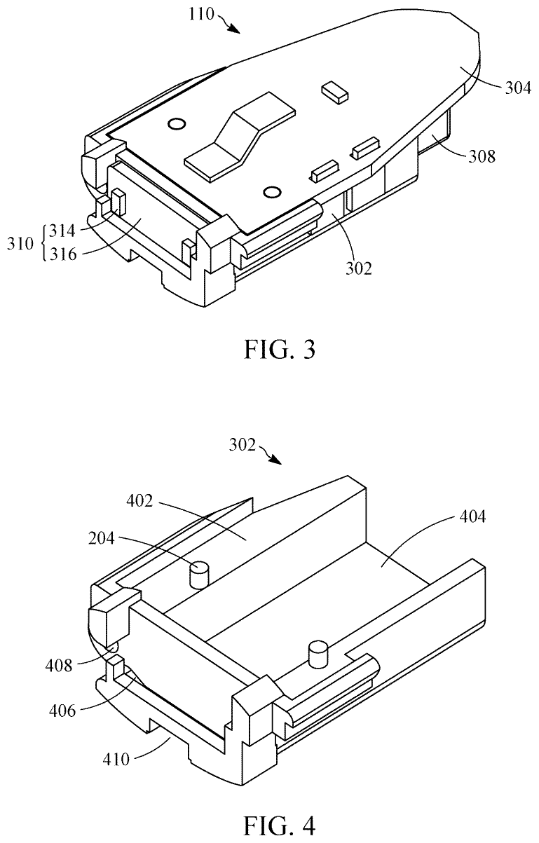

Referring to FIG. 3, the slotting unit 110 comprises a support base 302, a main circuit board (PCB) 304, a power source (a battery) 308, and one or more stimulation elements 314. The support base 302 is positioned inside the housing 104. The PCB 304, the battery 308 and a board 310 having the one or more stimulation elements are mounted over the support base 302. The battery 308 and the one or more stimulation elements 314 are in electrical connection with the main circuit board (PCB) 304. The board 310 having the one or more stimulation elements 314 is arranged in the light emitting direction of the one or more stimulation elements. The board 310 is connected to the main circuit board through the support base.

Referring to FIG. 3 to FIG. 5, the support base 302 is provided with a first card slot 402 on the side wall of the support base 302. The first card slot 402 is provided with a positioning guide rod 204. The main circuit board 304 has a positioning hole 318 for inserting the positioning guide rod 204. The positioning guide rod 204 and the positioning hole 318 helps in the alignment of the main circuit board 304 and the support base 302. The first card slot 402 is used to improve the positioning and disassembly between the main circuit board 304 and the support base 302.

In an embodiment of the present invention, the support base 302 and the main circuit board 304 comprises a plurality of the positioning guide rods 204 and the positioning holes 318 for aligning the main circuit board 304 with the support base 302.

A second card slot 404 is provided at the bottom of the first card slot 402. The second card slot 404 is used to accommodate the battery 308. The main circuit board 304 and the battery 308 are arranged along the thickness direction of the scoop body 102. The thickness of the slotting unit 110 is designed so as the housing 104 has enough space to accommodate the main circuit board 304 and the battery 308.

Referring to FIG. 4, the positioning guide rods 204 are two in number located at opposite side walls of the support base 302. The second card slot 404 is located between the two side walls of the support base 302. When the main circuit board 304 is installed over the first card slot 402, the main circuit board 304 covers the opening of the second card slot 404 and the main circuit board 304 aligns with the support base 302 using the positioning guide rods 204 and positioning holes 318. The battery 308 is fixed by clamping, thereby maintaining the position stability of the battery.

The end of the support base 302 facing light emitting plate 108 is provided with a third card slot 406. The third card slot 406 is used for inserting an auxiliary circuit board 316. The auxiliary circuit board 316 may include a sub-circuit board or auxiliary circuit board 316. The one or more stimulation elements 314 are electrically connected to the auxiliary circuit board 316. The one or more stimulation elements 314 are installed on the auxiliary circuit board 316 at intervals.

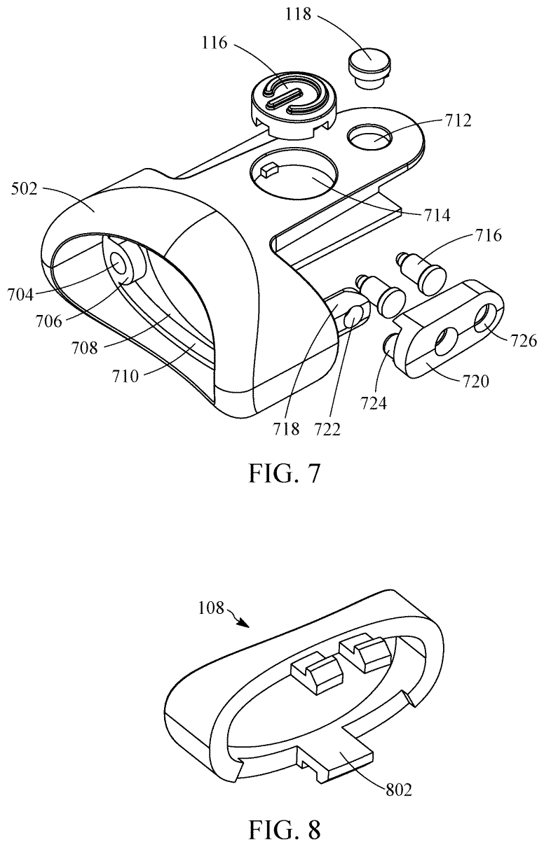

Referring to FIG. 4, FIG. 6 and FIG. 7, the housing 104 is provided with a guide column 602 having a hole for a screw. The slotting unit 110 has a first mounting hole 408 corresponding to the position of the guide column 602. The front cover unit 502 has a second mounting hole 704 at a position relative to the first mounting hole 408, and the guide column 602. A locking member or a screw is used that passes through the second mounting hole 704 and the first mounting hole 408, the locking member gets locked into the installation guide column 602. The guide column 602 is provided in the housing 104 of the scoop body 102.

The second mounting hole 704 is provided in the front cover unit 502. The inner wall of the front cover unit 502 is provided with a fixing sheet 706 on to which the second mounting hole 704 is provided. The locking member can be a screw, a bolt etc. The first cover unit 502, the slotting unit 110 and the scoop body 102 are fixed using the locking member. The front cover unit 502, the support base 302 and the scoop body 102 are connected to facilitate the connection between the scoop body 102 and one or more stimulation elements.

Referring to FIG. 8, a hook 802 is provided on the light emitting plate 108, the hook 802 gets fitted into corresponding slot or a clamping position 708 in the front cover unit 502. The slot or clamping position 708 can be groove or a step or like. The light emitting plate 108 and the front cover unit 502 can be detachably connected by the engagement of the hook and the engagement position.

In alternative embodiment, the hook 802 of the light emitting plate 108 can be fixed in a blocking sheet 710 provided on the inner side wall of the front cover unit 502 or in relief groove 410 provided on the support seat.

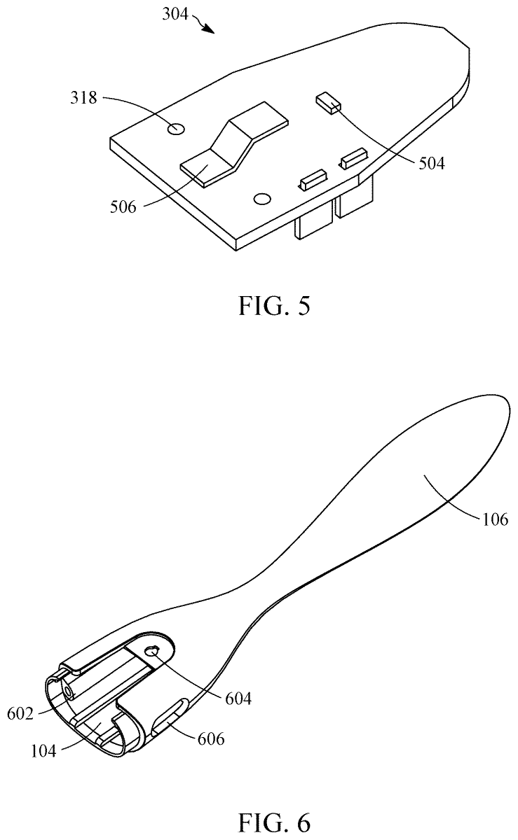

Referring to FIG. 5, FIG. 6 and FIG. 7, a control switch 116 and a light guide column 118 are electrically connected to the main circuit board 304. The front cover unit 502 is provided with a first hole 712 and a second hole 714 spaced apart from each other. The housing section 104 in the scoop body 102 has a third hole 604, relative to the corresponding position of the first hole 712. An indicator light 504 is disposed on the main circuit board 304 at a position relative to the third hole 604. The indicator light 504 is connected to the main circuit board 304. The light guide column 118 is installed in the first hole 712 and passes through the third hole 604.

A conductive spring sheet 506 is disposed on the main circuit board 304 at a position corresponding to the second hole 714. The control switch 116 is installed in the second hole 714 can abut against the conductive spring sheet 506.

The therapeutic applicator 100 is turned on or off by controlling the control switch 116. The light indicator 504 indicates whether the therapeutic applicator is on or off. The light emitted by the indicator light 504 is irradiated into the light guide column 118 and diffuses outside the scoop body 102.

Referring to FIG. 2, the therapeutic applicator further comprises a charging needle or charging pin 716 mounted on the scoop body 102. The charging pin 716 is electrically connected to the main circuit board 304. The charging pin 716 is connected to the battery 308 through the main circuit board.

Referring to FIG. 7, the front cover unit 502 is provided with a mounting arm 718 and a mounting member 720 connected to the mounting arm 718. The mounting arm 718 has a socket 722 in which an insertion rod 724 of the mounting member 720 gets inserted. The mounting member 720 is provided with a mounting hole 726 for mounting the charging pin 716. The mounting member 720 has two mounting holes 726 that can be used as positive and negative electrode respectively. The housing 104 in the scoop body 102 is provided with an opening for installation. The mounting member 720 extends into the avoidance hole 606, so that the charging needle 716 can extend out to the scoop boy 102 to achieve charging.

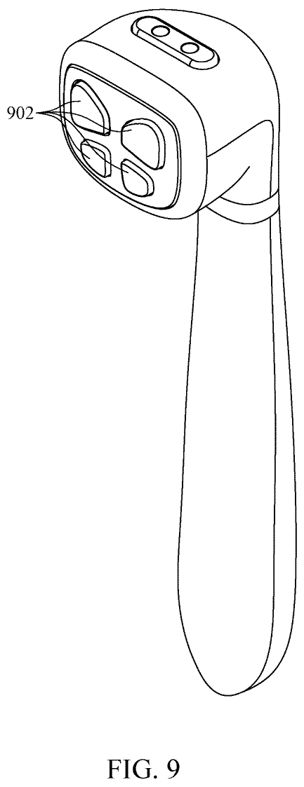

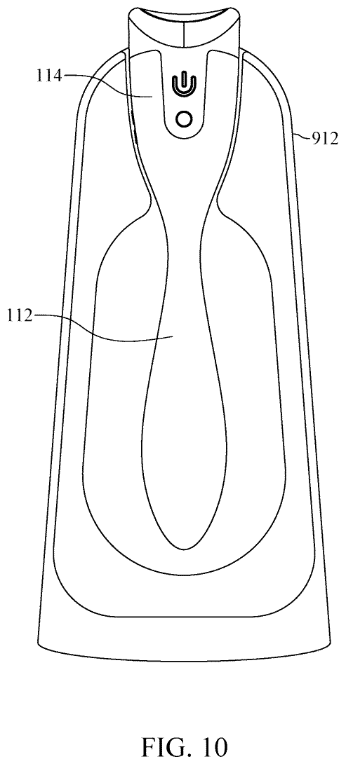

Referring to FIG. 9, a therapeutic applicator with a micro-current element 902 is provided. The micro-current element 902 is provided on the face of the front cover unit. The micro-current element is a butterfly-shaped metal piece. The micro-current element provides an electrical effect to the skin of the user.

Referring to FIG. 10, a charging station 912 is shown for charging the therapeutic applicator 100. The therapeutic applicator 100 is suspended on the charging station and the charging station is used to charge the therapeutic applicator. The charging station can charge the therapeutic applicator by hanging the therapeutic applicator in the charging station, such that the charging pin of the therapeutic applicator gets in contact with the charging unit on the charging station. The stimulation section 114 is at the upper end of the charging station and the scoop section 112 is at the lower and of the charging station.

In an embodiment of the present invention, a therapeutic applicator is provided. The therapeutic applicator comprises a scoop body having a first end and a second end, wherein the first end of the applicator is used for scooping and applying a cosmetic. The scoop body has a housing at the second end in which a slotting unit is inserted. The slotting unit has a support base, a PCB, at least one stimulation element in electrical connection with the PCB, and a power source. The at least one stimulation element is selected from a group consisting of Light Emitting Diodes (LEDs), lasers, heating elements, cooling elements, vibration elements, electrodes, micro-current element and combinations thereof. The scoop body at its applicator end may comprise one or more sensors to sense the pH and to monitor one or more parameters of the skin of a user. The one or more parameters include but are not limited to temperature of cosmetic, skin temperature, treatment time, skin pH etc. The therapeutic applicator communicates the monitored one or more parameters to an external communication device. The external communication device may have an app or programmable interface that determines a treatment regime for the corresponding portion of the user's skin. The external communication device communicates the determined treatment regime to the therapeutic applicator. The therapeutic applicator may comprise a processing unit or a programmable controller that activate and control one or more stimulation elements in therapeutic applicator based on the feedback received from the external communication device.

The applicator portion or the scoop portion of the therapeutic applicator may have thermal regulating elements, such as a heating element, a cooling element or a Peltier element. The use of thermal regulating element has specific advantages while using the cosmetic. The sensors in the applicator end can sense the temperature of the cosmetic and accordingly the micro-controller can control or activate the thermal regulating element for the treatment of the cosmetic.

For enabling pre-treatment of the user's skin before applying the cosmetic, the therapeutic applicator may have abrasive element or a brush or a rotating brush to pre-treat the skin.

The housing of the therapeutic applicator is covered with the front cover unit. The front cover unit fits over the housing and covers the slotting unit. The open end of the front cover unit is curved shape so that the therapeutic applicator can move on the user's skin conveniently.

The front cover unit has a micro-current element fixed at the end that touches the user's skin. The micro-current element is used to provide current therapy to the user.

In some embodiments, the therapeutic applicator is provided with automatic activation, such that the device is switched on automatically when a user is holding the device and the applicator part of the device touches the cosmetic or the skin of the user.

In another embodiment of the present invention, a therapeutic applicator is provided. The therapeutic applicator comprises a scoop body having a first end and a second end. The first end has an applicator for scooping and applying a cosmetic. The second end of the applicator has a housing in which a slotting unit is inserted. The slotting unit has at least one stimulation element. The therapeutic applicator may comprise one or more of a heating element, a cooling element or a Peltier element at the first end or the second end. The at least one stimulation element is selected from group consisting of Light Emitting Diodes (LEDs), lasers, heating elements, cooling elements, vibration elements, electrodes, micro-current element and combinations thereof.

The slotting unit comprises a support base, a Printed Circuit Board (PCB) installed on a first side of the support base, and the at least one stimulation element is in electrical connection with the Printed circuit board; a power source provided at a second side of the support base is in electrically connection with the PCB.

The scoop body at its applicator end may comprise one or more sensors to sense the pH and to monitor one or more parameters of the skin of a user. The one or more parameters include but are not limited to temperature of cosmetic, skin temperature, treatment time, skin pH, etc. The therapeutic applicator communicates the monitored one or more parameters to an external communication device. The external communication device may have an app or programmable interface that determines a treatment regime for the corresponding portion of the user's skin. The external communication device communicates the determined treatment regime to the therapeutic applicator. The therapeutic applicator may comprise a processing unit or a programmable controller that activates and controls one or more stimulation elements in the therapeutic applicator based on the feedback received from the external communication device.

In another embodiment of the present invention, a method for providing skin therapy to a user is provided. The method uses the therapeutic applicator device of FIGS. 1 to 10. The therapeutic applicator has an applicator end for scooping and applying a cosmetic and one or more stimulation elements to provide therapy to user. The method involves applying a cosmetic to the user using the applicator and providing therapy to the user using one or more stimulation elements present in the therapeutic applicator. The therapeutic treatment that may be imparted to the user includes but are not limited to phototherapy, cooling, heating, laser treatment, massage, induction of micro-current. The applicator can treat the cosmetic using heating, cooling, or a Peltier element. The skin of the user can further be pre-treated by a brush, a rotating brush, or an abrasive element in the applicator before applying the cosmetic. The method may further involve sensing the one or more parameters related to skin and communicating the parameters to an external communication device. The external communication device can determine the treatment regime for the user and instruct the therapeutic applicator to activate and control a specific stimulation element.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapeutic applicator, comprising:
a scoop body having a first end and a second end, the first end has an applicator for scooping and applying a cosmetic;
a housing at the second end of the scoop body;
a slotting unit in the housing at the second end of the scoop body, the slotting unit comprises a support base, a PCB, at least one stimulation element in electrical connection with the PCB, and a power source.

2. The therapeutic applicator of claim 1, wherein the at least one stimulation element is selected from a group consisting of Light Emitting Diodes (LEDs), lasers, heating elements, cooling elements, vibration elements, electrodes, micro-current element and combinations thereof.

3. The therapeutic applicator of claim 1, wherein the scoop body comprises one or more sensor to sense the pH and to monitor one or more parameters of the skin of a user.

4. The therapeutic applicator of claim 3, wherein the therapeutic applicator is in communication with an external communication device and communicates one or more parameters to the external communication device, the external communication device determines a treatment regime for the corresponding portion and communicates the treatment regime to the therapeutic applicator.

5. The therapeutic applicator of claim 1 further comprising a microcontroller to activate and control the at least one stimulation element based on the treatment regime determined for the corresponding portion.

6. The therapeutic applicator of claim 1, wherein the first end of the scoop body further comprises a heating element, a cooling element, a Peltier element or a combination thereof.

7. The therapeutic applicator of claim 1, wherein the first end of the scoop body comprises a brush, a rotating brush or an abrasive element to treat the skin before application of the cosmetic.

8. The therapeutic applicator of claim 1 further comprising: a front cover unit having a first end and a second end, the first end of the front cover unit locks into the second end of the scoop body to cover the slotting unit and the housing.

9. The therapeutic applicator of claim 8, wherein the second end of the front cover unit is curve shaped.

10. The therapeutic applicator of claim 1, wherein the therapeutic applicator turned on when a user hold the device and the first end of the scoop body is in contact with the cosmetic or skin of the user.

11. The therapeutic applicator of claim 1 further comprising a charging needle installed on the scoop body.

12. The therapeutic applicator of claim 1, wherein the center of the scoop body is circular in shape for holding by a user and diameter of center is less than the width at the first end and the second end of the scoop body.

13. A therapeutic applicator comprising:
a scoop body having a first end and a second end, the first end has an applicator for scooping and applying a cosmetic;
a housing at the second end of the scoop body;
at least one stimulation element in the housing at the second end of the scoop body;
wherein the first end of the scoop body comprises a thermal regulating element.

14. The therapeutic applicator of claim 13, wherein the at least one stimulation element is selected from a group consisting of Light Emitting Diodes (LEDs), lasers, heating elements, cooling elements, vibration elements, electrodes, micro-current element and combinations thereof.

15. The therapeutic applicator of claim 13, wherein the thermal regulating element comprises a heating element, a cooling element, a Peltier element or a combination thereof.

16. The therapeutic applicator of claim 13, wherein the scoop body comprises one or more sensor to sense the pH and to monitor one or more parameters of the skin of a user, the therapeutic applicator communicates the one or more parameters of the skin to an external communication device.

17. The therapeutic applicator of claim 16, wherein the external communication device determines a treatment regime for the corresponding portion and communicates the treatment regime to the therapeutic applicator.

18. The therapeutic applicator of claim 17 further comprising a microcontroller to activate and control the at least one stimulation element based on the treatment regime determined for the corresponding portion.

19. The therapeutic applicator of claim 13 further comprising a micro current element.

20. A method for providing skin therapy to a user, the method comprising:
providing a therapeutic applicator having an applicator for scooping and applying a cosmetic and a first end, and at least one stimulation element at the second end;
applying the cosmetic to a user's skin;
using the stimulation element to provide therapeutic effect to the user's skin.

21. The method of claim 20, wherein the at least one stimulation element is selected from a group consisting of Light Emitting Diodes (LEDs), lasers, heating elements, cooling elements, vibration elements, electrodes, micro-current element and combinations thereof.

22. The method of claim 20, wherein the therapeutic treatment includes but is not limited to phototherapy, cooling, heating, laser treatment, massage, induction of micro-current.

23. The method of claim 20 further comprising: sensing the pH of the skin and monitoring one or more parameters of the skin by using one or more sensor.

24. The method of claim 20, wherein the skin of the user is pre-treated by a brush, a rotating brush or an abrasive element in the applicator before applying the cosmetic to the skin.
